(19) United States
(12) Patent Application Publication
(10) Pub. No.: US 2008/0172022 A1
(43) Pub. Date: Jul. 17, 2008

(54) INFUSION CONTAINER

(75) Inventors: Ramon E. Poo, Miami, FL (US); Camillo Ricordi, Miami, FL (US); Felipe Echeverri, Miami, FL (US)

Correspondence Address: AKERMAN SENTERFITT, P.O. BOX 3188, WEST PALM BEACH, FL, 33402-3188, US

(73) Assignees: Biorep Technologies, Inc., Miami, FL (US); University of Miami, Miami, FL (US)

(21) Appl. No.: 11/622,232

(22) Filed: Jan. 11, 2007

(51) Int. Cl. A61B 19/00

(52) U.S. Cl. 604/408; 604/415

(57) ABSTRACT

An infusion container includes a main body portion with side walls, and top and bottom portions. The bottom portion has an included angle, the included angle being between 30-120 degrees. The included angle serves to channel cells to be infused into a patient out of the container, to reduce waste and damage to the cells. A method of infusion is also disclosed.

### BACKGROUND OF THE INVENTION

The infusion of donor cells such as islet cells requires that the cells be maintained in some type of container prior to infusion into the patient. Typically, the cells are placed in solution into a flexible sterile container. An outlet port of the container is connected to a flexible conduit which delivers the cells and solution to a catheter for infusion. Some cells become collected at the horizontal bottom of the square or rectangular shaped container, and do not pass through the outlet port. Valuable cells are thereby lost in the infusion process, or time is lost as the container is manipulated to cause the cells to pass from the container. The loss of time and manipulation of the cells increases the risk of injury to the cells.

### SUMMARY OF THE INVENTION

An infusion container comprises a main body portion comprising side walls, and top and bottom portions. The bottom portion has an included angle, the included angle being between 30-120 degrees. In another aspect, the included angle is between 40-100 degrees. In yet another aspect, the included angle is between 50-80 degrees. The included angle can be about 60 degrees. An outlet can be provided through which the contents exit the container. The outlet can be of any suitable design.

The side walls can comprise a flexible material such that the container will be in the nature of a bag. The side walls can comprise a substantially transparent or translucent material. The container can comprise a material that is oxygen permeable. The container can comprise a material that is carbon dioxide permeable.

The container can comprise at least one port for the introduction of material into the bag. The at least one port can be selected from the group consisting of a luer lock, a spike entry port, and an injection port.

A method of infusing cells according to the invention comprises the steps of providing an infusion container, the infusion container having a main body portion with side walls, and top and bottom portions, the bottom having an included angle, the included angle being between 30-120 degrees. A liquid solution and cells to be infused into a patient are placed into the container. The cells and solution are allowed to exit the container through an outlet in the bottom portion, whereby the cells will directed by the included angle to the outlet. The infusion container may also be used to transport and culture islet cells in the incubator due to its high gas permeability characteristics.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 is a side elevation of an infusion container according to the invention.

FIG. 2 is a side elevation of an infusion container according to an alternative embodiment.

### DETAILED DESCRIPTION OF THE INVENTION

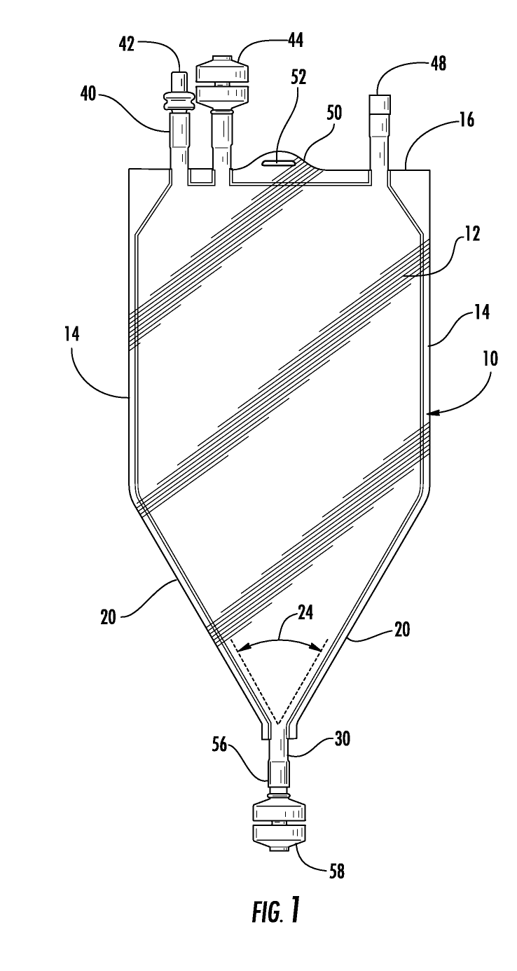

There is shown in FIG. 1 an infusion container 10 according to the invention. The container 10 can comprise side walls such as opposing front and rear side main body panels 12 joined at side edges 14 thereof. A tubular side wall or seamless construction is also possible. The container 10 further has a top portion 16 and bottom portion 20. The bottom portion 20 defines an included angle 24. The included angled 24 permits cells and other solid and viscous materials to readily drain from the infusion container 10 through a suitable outlet port 30. The outlet port 30 is positioned substantially at the vertex of the included angle 24.

The shape and capacity of the container can be varied, provided that the bottom of the container has the included angle construction of the invention. The sides of the included angle 24 need not be linear, curved or sloping sides are possible, provided that the outlet port is below the sides when the infusion container is in use and that the surface is shaped so as to permit cells to effectively leave the container. Therefore, the term vertex as used herein generally refers to the point where the sides of the bottom portion 20 converge, which will generally be the lowest point of the container when it is positioned upright.

The included angle 24 can be any suitable angle which facilitates the egress of material from the infusion container 10. In one embodiment, the included angle is between about 30-120 degrees. In another embodiment, the included angle is between about 40-100 degrees. In still another embodiment, the included angle 24 is between about 50-80 degrees. The included angle can be between about 55-65 degrees, and can be about 60 degrees.

The dimensions and capacity of the container can be varied. In one aspect, the container has a capacity of between about 500 ml and 1 L, and for pancreatic cell transplantation purposes a capacity of about 600 ml is useful.

The container 10 can be constructed from a material that is permeable to oxygen and to carbon dioxide. In this manner, cells within the container can receive oxygen and carbon dioxide can be removed from the container 10. This will facilitate the continued viability of the cells. The permeability of the material to oxygen is selected to deliver oxygen at least at the minimum consumption rate that is required by the cells contained within the containers, and will depend on the amount of cells that are in the container 10. In the particular case of pancreatic islet cells, for example, this rate is about 7.2×10⁻⁸ mol/(cm³·s) at 37° C. The rate of transfer of gases into or from the container can be varied for the oxygen demand and number of cells intended to be placed within the container, by suitable methods such as changing the thickness of the container material, the composition of the container material, and the size and geometry of the container. An oxygen permeability of between about 50-500 cm³/(100 in²*24 hr*atm@25° C.) is desirable for many types of cells, and an oxygen permeability of between about 98-293 cm³(100 in/²*24 hr*atm.@25° C.) can be useful. Children and adults, as well as differently sized people in general, can require differing amounts of cells for an islet cell transplantation, and the more cells that are placed within a same size container, generally the greater the oxygen permeability that will be required. Since islet cells are considered to be one of the most oxygen demanding cells (since they are metabolically active), then designs that are satisfactory for these types of cells should also be satisfactory for many other kinds of biological cell types.

The container is preferably transparent or translucent in part to permit visual inspection of the contents. A suitable container material is ES-4000™, a flexible vinyl film manufactured by Solvay Draka Inc. of Commerce, Calif. ES-4000 has an O₂ transmission of 146 cm³/(100 in²*24 hr*atm@25° C.). The container material can have any suitable thickness. The thickness of the container material will affect flexibility and the permeability to oxygen and carbon dioxide, and the thickness can be adjusted accordingly. In one aspect the material thickness is about 0.015 inches.

The container 10 can be provided with various ports for the introduction of materials into the container. One such port is a female Luer lock 40. The female Luer lock 40 is adapted to connect to a male fitting for the introduction of material into the container 10, and can be covered by a cap 42 until ready for use. Another suitable port is the spike entry port 44. Yet another is an injection cap 48. The injection cap 48 has an elastomeric material as a covering which permits a syringe needle to be placed through the material to introduce materials into the container 10. The elastomeric material seals when the needle is withdrawn. Other ports or apparatus used with infusion containers are possible. A tab 50 with a slit 52 or other suitable structure can be provided to permit the container 10 to be hung from a support. Various valve and port constructions can similarly be provided with the outlet port 30, such as the spike outlet port 56 with cap 58, to control egress of the contents of the container through the outlet.

The infusion container 10 can be used for other purposes due to its high gas permeability characteristics. The infusion container 10 can also be used to transport and culture islet cells in the incubator, and the permeability of the container will permit gas exchange through the container so as to foster cell maintenance and growth.

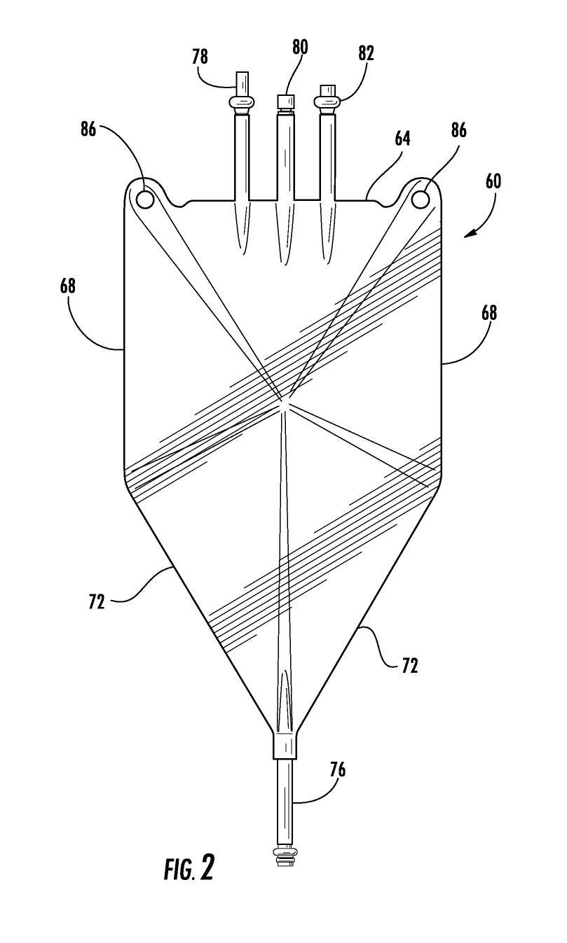

An alternative container design is shown in FIG. 2. The container 60 includes top 64, sides 68, and bottom 72. The bottom 72 is formed from curved sides that slope to the outlet port 76. A plurality of inlet ports 78, 80 and 82 can be provided at the top 64. Hanging rings 86 can be provided at sides of the top 64 to permit the hanging of the container from a suitable support.

The permeability and size of the container can be selected so as to provide the gas exchange necessary for maintenance and growth if desired for the intended contents. In one embodiment the oxygen permeability, and volume of the container are selected such that the flow rate of oxygen into the container is sufficient to satisfy the oxygen demand of the cells inside the container. The oxygen permeability in one embodiment is selected using atmospheric pO₂ at a given temperature.

The following example will be provided for the case of pancreatic cell transplantation, however, it will be appreciated that the invention has utility for a variety of different kinds of cells and organs. In this example, in order for islet cells to survive inside the infusion container, the oxygen transmission rate through the container film has to be greater than the oxygen consumption rate of the islet cells. It has been shown that a pO₂<40 mmHg leads to hypoxia in islet cells. Therefore, it is assumed that a 50% higher pO₂ inside the container (60 mmHg pO₂) will be sufficient to maintain the cells. Also, it is assumed that outside the container the pO₂ is 142 mmHg (21% of 760 mmHg after accounting for 5% CO₂ and moisture at 37.0° C. inside an incubator).
Therefore, the ΔpO₂ is: p0₂_{_{incubato\dot{r}}}:=142 mmHg
   ΔpO₂:=pO₂_{_{incubato r̅}}=pO₂ bag
   ΔpO₂:=pO₂_{_{incubato r̅}}=pO₂ bag
   ΔpO₂=82 mmHg
      Both the transmission and oxygen consumption parameters need to be adjusted at the same (actual) temperature. The oxygen transmission rate for ES-4000 film is given at 25.0° C. Therefore, this calculation is an approximation.

Starting with the ES-4000 film oxygen transmission rate:

$$TR := 146 \cdot \frac{{cm}^{3}}{100 \cdot {in}^{2} \cdot 24 \cdot hr \cdot atm} @ 25.0 C .$$

It is known that for an ideal gas, the standard molar volume is the volume that is occupied by one mol of substance (in gaseous form) at standard temperature and pressure (STP) of 273.15 K (H₂O freezing temperature) and 101325 Pa (1 atm). It is 22.42 L/mol and is directly related to the universal gas constant R in the ideal gas law.

Therefore, the volume occupied by one mol of a gas at 25.0° C. and 1 atm is:

$$T := 298.15 K$$

$$R := 8.314570 \cdot \frac{N \cdot m}{K \cdot mol}$$

$$P := 101325 \frac{N}{{m}^{2}}$$

$$MV = 2.447 \times {10}^{4} \frac{{cm}^{3}}{mol}$$

$$MV := \frac{R \cdot T}{P}$$

This factor can be used to turn cm³ of oxygen to mol of oxygen at the given temperature and pressure. Therefore we can express the oxygen transmission rate of the container in terms of

mol/cm² sec mmHg

$$Tx := \frac{TR}{MV}$$

$$Tx = 1.538 \times {10}^{- 13} \frac{mol}{{cm}^{2} \cdot s \cdot mmHg}$$

The transmission rate can be multiplied times the ΔpO₂ (based on the assumption) to express the transmission rate in terms of mol/cm²·sec

$$Tx := \frac{TR \cdot Δ p O 2}{MV}$$

$$Tx := 1.261 \times {10}^{- 11} \frac{mol}{{cm}^{2} \cdot sec}$$

The infusion container can have a surface area of (one side):

A:=206·cm²

The transmission rate of the container then becomes (assuming oxygen transmission occurring throughout top and bottom film):

$${IB}_{OTR} := \frac{Tr \cdot Δ p O 2 \cdot A}{MV} \cdot 2$$

$$Both sides$$

$${IB}_{OTR} = 5.195 \times {10}^{- 9} \frac{mol}{s}$$

This oxygen transmission rate can be compared to the oxygen consumption rate of islet cells. For a preparation to be “transplantable”, there have to be approximately 4,000-6,000 IEQ/kg. This means (assume a 70 kg average person), to treat diabetes, anywhere from 280,000-420,000 IE need to be transplanted. Assuming a 350,000 IE preparation is placed inside the container.

IE:=30000(

One IE islet tissue mass is equivalent to a spherical islet of 150 μm in diameter

$$r := 75 \cdot {10}^{- 6} \cdot m$$

$${V}_{IE} := \frac{4}{3} \cdot π \cdot {r}^{3}$$

$${V}_{IE} = 1.767 \times {10}^{- 6} {cm}^{3}$$

Therefore the volume of the islet cell preparation would be:

Vₚᵣₑₚ:V_{IE}IE

Vₚᵣₑₚ=0.53 cm³

From literature, the maximum oxygen consumption rate of islet cells is:

$${IC}_{OCR} := 7.2 \cdot {10}^{- 8} \cdot \frac{mol}{{cm}^{3} \cdot sec}$$

$${Prep}_{OCR} = 3.817 \times {10}^{- 8} \frac{mol}{s}$$

Therefore the total of 300,000 IE would have an O₂ consumption rate of:

Prep_{OCR}:=IC_{OCR}·Vₚᵣₑₚ

The infusion container oxygen transmission rate can be compared to the islet cell preparation oxygen consumption rate, as follows:

$${IB}_{OTR} = 5.195 \times {10}^{- 9} \frac{mol}{s}$$

$${Prep}_{OCR} = 3.817 \times {10}^{- 8} \frac{mol}{s}$$

The preparation consumes oxygen at a higher rate than the container can transmit. Since IB_{OTR}<Prep_{OCR}, it means that if 300,000 IE are placed inside the infusion container, the islet cells will start having hypoxic or anoxic cores and they will die. Accordingly, only about 40,000 IE can be safely placed inside the infusion container for cell culturing.

A method of infusing cells according to the invention comprises the steps of providing an infusion container, the infusion container having a main body portion with side walls, and top and bottom portions, the bottom portion having an included angle, the included angle being between 30-120 degrees. A liquid solution and cells to be infused into a patient are placed into the container. The cells and solution are allowed to exit the container through an outlet at a vertex of the bottom portion, whereby the cells will directed by the included angle to the outlet. The infusion container 10 may also be used to transport and culture islet cells in the incubator due to its high gas permeability characteristics.

This invention can be embodied in other forms without departing from the spirit or essential attributes thereof and, accordingly, reference should be had to the following claims rather than the foregoing specification as indicating the scope of the invention.

1. An infusion container, comprising:
a main body portion comprising side walls, and top and bottom portions;
wherein the bottom portion has an included angle having a vertex, the included angle being between 30-40 degrees;
at least one outlet, the outlet being provided substantially at the vertex of said included angle;
said container comprising a material having an oxygen permeability of between about 50-500 cm³/(100 in²*24 hr*atm @25° C.).

2. (canceled)

3. (canceled)

4. (canceled)

5. The infusion container of claim 1, wherein the side walls comprise a flexible material.

6. The infusion container of claim 1, wherein the side walls comprise a substantially translucent material.

7. The infusion container of claim 1, wherein the side walls comprise a substantially transparent material.

8. The infusion container of claim 1, wherein the container comprises a material that is oxygen permeable.

9. The infusion container of claim 1, wherein the container comprises a material that is carbon dioxide permeable.

10. The infusion container of claim 1, wherein the container comprises at least one port for the introduction of material into the container.

11. The infusion container of claim 1, wherein said at least one port is at least one selected from the group consisting of a Luer lock, a spike entry port, and an injection port.

12. (canceled)

13. The infusion container of claim 1, wherein said container is constructed of a material having an oxygen permeability of between about 98-293 cm³/(100 in²*24 hr*atm @25° C.).

14. A method of infusing cells, comprising the steps of:
providing an infusion container, the infusion container comprising a main body portion comprising side walls, and top and bottom portions, the bottom portion having an included angle, the included angle being between 30-40 degrees, and an outlet at a vertex of the included angle, wherein said container comprises a material having an oxygen permeability of between about 50-500 cm³/(100 in²*24 hr*atm @25° C.);
placing into the container a liquid solution and cells to be infused into a patient;
allowing the cells and solution to exit the container through the outlet, whereby the cells will directed by the included angle to the outlet.
